(19) United States
(12) Patent Application Publication
(10) Pub. No.: US 2025/0049661 A1
(43) Pub. Date: Feb. 13, 2025

(54) FRAGRANCE COMPOSITIONS INCLUDING GAS FERMENTED ETHANOL AND METHODS OF MAKING

(71) Applicants: Mariano Agustin RODRIGUEZ-FRATICELLI, Versoix (CH); Jose Maria VELAZQUEZ MENDOZA, Nyon (CH); Coty Inc., New York, NY (US)

(72) Inventors: Mariano Agustin Rodriguez-Fraticelli, Versoix (CH); Jose Maria Velazquez Mendoza, Nyon (CH)

(21) Appl. No.: 18/710,987

(22) PCT Filed: Dec. 21, 2022
(86) PCT No.: PCT/US2022/082135

§ 371 Date: May 16, 2024

(30) Foreign Application Priority Data
Dec. 22, 2021 (FR) FR2114220

(51) Int. Cl. A61K 8/34; A61Q 13/00

(52) U.S. Cl. CPC A61K 8/34; A61Q 13/00; A61K 2800/85

(57) ABSTRACT

Various aspects relate to a composition. The composition includes a carbon-emissions-derived ethanol distillate and a fragrance component. The carbon-emissions-derived ethanol distillate is a distillate comprising ethyl alcohol and the composition is formulated as a cosmetic or a perfume.

### CLAIM OF PRIORITY

This patent application claims the benefit of priority to French Application Serial No. 2114220, filed Dec. 22, 2021, which is incorporated by reference herein in its entirety.

### BACKGROUND

Traditional ethanol employed in the perfume industry comes from agricultural crops such as sugar beet, corn and sugar cane. In all cases, yeasts are used to ferment the sugars from the substrates and produce ethanol and byproducts. Subsequent separation from the yeast brew and distillation will ensure that the ethanol is extracted from all other non-volatiles and volatile molecules alike. The result is a safe-to-use and more importantly neutral grade ethanol that can be used to formulate consumer products.

### SUMMARY

Various aspects relate to a composition. The composition includes a carbon-emissions-derived ethanol distillate and a fragrance component. The carbon-emissions-derived ethanol distillate is a distillate comprising ethyl alcohol and the composition is formulated as a cosmetic or a perfume.

Various aspects relate to a method of making a composition. The composition includes a carbon-emissions-derived ethanol distillate and a fragrance component. The carbon-emissions-derived ethanol distillate is a distillate comprising ethyl alcohol and the composition is formulated as a cosmetic or a perfume. The method includes fermenting a carbon emission to produce a carbon-emissions-derived ethanol composition. The method further includes distilling the carbon-emission-derived ethanol composition to produce a carbon-emissions-derived ethanol distillate. The method further includes mixing the carbon-emissions-derived ethanol distillate with the fragrance component.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

In the perfume industry, ethanol is considered mainly a solvent whose properties are mostly functional. The odor of the ethanol is a negative parameter, and thus is neutralized during the distillation process. However, every ethanol has a distinctive smell. Some volatile residues remain in the final ethanol distillate due to the close boiling point to ethanol. Others are reduced to sub-micron level, but due to low olfactive detection threshold are still perceived by the human nose.

These volatile residues originate mainly from the raw material (fruits, crops, etc.), the fermentation process, and the distillation process. Additional processing like wine-making process or the extraction and conversion of starchy crops to sugars could have an impact on remaining volatile residues. As well, instabilities due to oxidation could lead to further volatile residues. It is therefore evident that different raw materials and processes will lead to different olfactive profiles in ethanol.

Further evidence is that modifying the properties of ethanol to improve perfumes well is sought for in the patent literature. Sometimes achieved by selecting specific raw materials like grape-fermented ethanol (U.S. Pat. No. 7,576,045), using the residue produced in wine-manufacturing to purify the ethanol (EP0196340) or the addition of rice liquor in order to mask the ethanol odor (JPH06104605B2). The observed benefits were the elimination of the pungency of ethanol, clarity of the fragrance smell, storage stability and the elimination the odor “typical” of ethanol. However, it is understood that some additional odors may be associated with ethanol such as a grape-fermented ethanol that are still perceivable. Therefore, it can be beneficial to produce compositions free of grape-fermented ethanol (e.g., vinal alcohol).

The potential for biologically fixating carbon that otherwise would end up in the atmosphere has great potential in reducing the impact of global warming. Since recently, ethanol has been produced industrially by fermenting carbon emissions (mainly carbon monoxide, carbon dioxide, and hydrogen) in tanks. Carbon emissions can be captured from industrial off-gases, cleaned up, compressed and injected into a fermentation tank. Acetogenic bacteria transform these off-gases into ethanol and by-products such as sulfur compounds. Further separation, distillation and dehydration produce a highly concentrated ethanol (>99%) to be used in the fuel industry. In order to produce a high purity alcohol for cosmetic and perfumery uses, further distillation steps (purification) are. During the purification step, both heads, representing highly volatile materials, and tails, representing low volatile materials, are further reduced to achieve a neutral-grade alcohol. A crude-ethanol purification system may include a three-distillation column process. First an extractive distillation unit where water and ethanol are put in contact, and where low volatile impurities like acetaldehyde are removed. Then the purified ethanol from the extractive column may be fed into the rectifying column which has a series of trays, where the ethanol gets successively concentrated. A small part of heads can be removed from the top, whereas fossil oils (mainly propanol) are drawn off before the trays where the pure ethanol is concentrated. The final concentration of ethanol is achieved at this column. Finally, a demethylizer column where the main focus is to remove methanol.

An aspect of this disclosure is to describe a composition including a purified carbon-emissions based ethanol and at least one fragrance composition, which has better olfactive stability over time and enhances the olfactive character of formulated fragrances.

Therefore, according to an aspect of the instant disclosure, a composition includes a carbon-emissions-derived ethanol distillate and a fragrance component. The composition can be in a form selected from the group comprising an extract, eau de toilette, a creamy perfume, a bath preparation, an environmental fragrance, or a mixture thereof. If the composition is a perfume or cosmetic, it can be in a liquid form, a gelled form, a viscous form, solid form, or a mixture thereof.

The composition can include from about 50 wt % to about 95 wt % of the carbon-emissions alcohol distillate, 70 wt % to 95 wt %, less than, equal to, or greater than about 50 wt %, 55, 60, 65, 70, 75, 80, 85, 90, or about 95 wt %. While the carbon-emissions alcohol distillate mainly includes ethanol, it is possible for the carbon-emissions alcohol distillate to also include small amounts of volatile and non-volatile by-products.

The carbon-emissions alcohol distillate is a fermentation product. However, as opposed to conventional ethanol, used in cosmetic compositions, the ethanol present in the carbon-emissions alcohol distillate is the product of a gas fermentation process that uses a carbon-emissions gas stream as a feedstock. The carbon-emission gas stream can include CO, CO₂, H₂, or a mixture thereof. A common feedstock is syngas.

The overall gas fermentation process can be broadly divided into four steps: (1) accumulation or generation of syngas; (2) gas pretreatment; (3) gas fermentation in a bioreactor; and (4) product separation.

### Generation of Syngas

The versatility of acetogenic bacteria to ferment syngas of diverse compositions means virtually any carbonaceous materials can be gasified to generate the feedstock. When gasification is utilized, the carbonaceous material reacts with steam and air at an elevated temperature (600-1000° C.) and high pressure (>30 bar) to form syngas of variable composition (depending on input and process parameters). Although a small amount of energy input is required to heat the incoming feedstock to gasification temperature at the beginning of the process, at steady-state the process is self-sustaining. Excess heat generated from gasification can be used to generate steam for product distillation and/or electricity. Depending on the type of gasifier, the starting material may be subjected to drying, commination (size reduction), chipping, pelletization, torrefaction, pyrolysis and/or pulverization prior to gasification. Defined by how the reactor brings about contact with the feedstock and reactive gas, there are four main gasifier configurations: fixed/moving bed, fluidized bed, entrained flow, and transport flow. Fluidized bed gasifiers are currently the most commonly used biomass gasifiers due to their ease of up-scaling, isothermal operation conditions and high feedstock conversion efficiencies.

### Gas Pretreatment

In addition to the main constituents CO, H₂, CO₂, input gas streams can also contain impurities such as particulates, tar, aromatics grouped as benzene, toluene, ethylene, xylenes (BTEX) and naphthalene, sulfur compounds such as hydrogen sulfide (H₂S), carbonyl sulfide (COS), and carbon disulfide (CS₂), halogens such as chlorine and hydrogen fluoride (HF), and other potentially inhibiting gases such as ammonia (NH₃), nitric oxide and nitrogen dioxide (NOₓ), acetylene, oxygen (O₂), reactive oxygen species (ROS), and hydrogen cyanide (HCN). These are generated for example during the gasification process, pyrolysis or manufacturing and can be present in fluctuating quantities. A complete understanding of impurity species, their concentration fluctuations based on syngas input, process variables as well as installed treatment capacity is critical to maintain optimal productivity. In addition, monitoring impurity accumulation patterns within the fermentation is required to determine biological tolerance levels and the minimal inhibitory concentration (MIC) for designing economical treatment capacity. Understanding the effect of impurities could save treatment costs, however failure to do so can cause delays reaching full scale production capacity as shown in one large-scale syngas fermentation endeavor.

Even with gas-fermenting microorganisms' abilities to grow in the presence of low levels of impurities, some impurities necessitate near complete removal from an operational, biological and/or product specificity perspective. Particulates can be removed by cyclone separators and filters. Tars can be condensed and removed by quenching hot syngas, or, alternatively, can be reformed by heating at 800-900° C. using olivine, dolomite, and nickel compounds as catalysts, generating additional syngas.

Many contaminants including BTEX are lipophilic compounds that readily dissolve in the cytoplasmic membrane affecting membrane fluidity. Although polycyclic aromatics do not readily dissolve in aqueous phase, they can accumulate and negatively affect operations. Removal technologies for aromatics from gas are commercially available, and techniques to improve efficiency are still being reported. O₂'s toxicity above microoxic levels is particularly critical during inoculation of a bioreactor, when little biomass has accumulated to withstand introduction of aerobic conditions. O₂ can be tolerated in certain microoxic conditions. C. ljungdahlii has been shown to detoxify O₂ and ROS (likely via rubrerythrin and hydrogen peroxidases) and ethanol formation could actually be stimulated by exposure to O₂ (likely due to changes in AOR activity and co-factor metabolism). O₂ can be removed by passing the gas over various metal catalysts such as Pt, Pd, and Cu. Using biological co-culture for O₂ removal has also been described. Sulfur-containing impurities (e.g., H₂S and COS) can poison the metal based catalysts and require prior removal despite the microorganisms' ability to grow in their presence.

Acetylene, NOₓ and HCN are considered particularly troublesome as they are known to inhibit enzymes responsible for initial harvesting of energy from syngas. Cyanide binds to CODH, a key enzyme of the WLP. NO is a non-competitive inhibitor of hydrogenase activity while acetylene reversibly inhibits hydrogenases, which reduce ferredoxin for use in redox reactions. INEOS Bio has identified and reported HCN as a key contaminant that needs treatment from operation of their Vero Beach plant.

### Gas Fermentation

Treated syngas is next cooled and compressed then sparged into a bioreactor containing the gas-fermenting microorganisms in an aqueous medium. The fermenting microorganisms can be acetogenic bacteria or CI-fixing microorganisms, which have been demonstrated to convert gases containing CO2, CO, and/or H2 into products such as ethanol and isopropanol

For example, a microbial biomass including the acetogenic bacteria of CI-fixing bacteria comprises at least one suitable microorganism used as the biocatalyst of the fermentation process. For example, the microorganism may be selected from Escherichia coli, Saccharomyces cerevisiae, Clostridium acetobutylicum, Clostridium beijerinckii, Clostridium saccharbutyricum, Clostridium saccharoperbutylacetonicum, Clostridium butyricum, Clostridium diolis, Clostridium kluyveri, Clostridium pasterianium, Clostridium novyi, Clostridium difficile, Clostridium thermocellum, Clostridium cellulolyticum, Clostridium cellulovorans, Clostridium phytofermentans, Lactococcus lactis, Bacillus subtilis, Bacillus licheniformis, Zymomonas mobilis, Klebsiella oxytoca, Klebsiella pneumonia, Corynebacterium glutamicum, Trichoderma reesei, Cupriavidus necator, Pseudomonas putida, Lactobacillus plantar um, and Methylobacterium extorquens. In certain instances, the microorganism may be a Cl-fixing bacterium selected from Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drake i, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kiuvi. In a specific embodiment, the microorganism is a member of the genus Clostridium. In certain instances, the microorganism is Clostridium autoethanogenum.

There are a multitude of variables to account for during gas fermentation. Bioreactor design, agitation, gas composition and supply rate, pH, temperature, headspace pressure, oxidation-reduction potential (ORP), nutrients, and amount of foaming in the bioreactor all can contribute to the goal of improving selectivity and yield of the desired product (e.g., ethanol and butanol) as discussed below.

One major obstacle immediately present in gas fermentation is the low solubility of the gaseous substrates and combined with an efficient transfer of their masses into the liquid media. CO, H₂, and CO₂ are soluble to approximately 28 mg/L, 1.6 mg/L, and 1.7 g/L (293 K, 1 atm), respectively, compared to 900 g/L for glucose, a prevalent substrate for traditional fermentations. As gas-fermenting microorganisms consume the gas, substrate availability can become rate-limiting. Increasing flow of the substrate gas can lead to decreased yields of product per mole of carbon fed to the reactor, making reactor design and operation crucial. Continuous stirred tank reactors (CSTR) offer excellent mixing and homogenous distribution of gas substrates to the microorganisms and are most commonly employed at laboratory scale. However, the high power per unit volume required to drive the stirrer renders commercial scale operation economically challenging. Therefore, other less energy-demanding bioreactor designs such as bubble column, loop, and immobilized cell columns and their specific volumetric mass-transfer coefficients (k_{L}a) that describes the efficiency of which a gas can be delivered to a bioreactor have been investigated intensively and reviewed elsewhere.

Next to gas availability determined by reactor k_{L}a, the ratio and partial pressures of CO, H₂, and CO₂ also influence the product yield, production rate, and selectivity of a gas fermentation. CO and H₂ are sources of electrons/reducing equivalents for reducing CO₂ in the WLP and generating reduced products over acid products (e.g., ethanol vs. acetate). This product profile reflects the organism's requirement to maintain an internal energy balance that favors growth and is directly influenced by the gas composition and availability. As an example, productivity with C. ljungdahlii was improved from 38.4 g/L/d at 1 atmosphere to 360 g/L/d at 6 atmospheres. Another strategy to control the product profile is lowering the pH of the fermentation culture. This pH change can lead to a (reversible) shift from acidogenesis to solventogenesis allowing an increased production of ethanol and other highly reduced products.

Besides gas, medium composition also affects product yield and selectivity. Nutrient optimization has proven to be a process and species specific requirement. Media optimizations have been conducted for many acetogens including C. autoethanogenum, C. ljungdahlii, C. ragsdalei, C. aceticum, and M. thermoacetica. B vitamins and metals such as zinc, nickel, selenium, and tungsten, required as cofactors for certain enzymes in the central metabolism, are required for bacterial growth and affect product selectivity. For an industrial process it is critically important to keep media cost low, for example by eliminating yeast extract requirements, recycling nutrients and usage of industrial-grade bulk chemicals.

### Product Separation

Finally, product separation is required to separate the desired metabolic product (ethanol) from the fermentation broth. Distillation systems are common to separate lower boiling point products such as ethanol and acetone, but this is considered energy-intensive (and therefore potentially expensive), especially for low concentration products and products with high boiling points (e.g., butanediol). Other technologies to separate fermentation products from broth include liquid-liquid extraction, gas stripping, adsorption, perstraction, pervaporation, and vacuum distillation Each of these separation technologies has their own benefits and drawbacks, including potential fouling of membranes (perstraction and pervaporation) and substrate removal (gas stripping and liquid-liquid extraction). Liquid-liquid extraction is also an option for removing acetate from the fermentation broth of gas-fermenting acetogens.

The carbon-emissions alcohol distillate produced is combined with a fragrance component. The fragrance component is present in a range of from about 0.1 wt % to about 30 wt % of the composition, about 5 wt % to about 25 wt %, less than, equal to, or greater than about 0.1 wt %, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 wt % of the composition.

The fragrance component can include a material having an olfactive character classified as a green note, a fruity note, a spicy note, a citrus note, a floral note, or a combination thereof. The fragrance material can also be characterized according to its vapor pressure. For example, the vapor pressure can be in a range of from about 0.57 Torr to about 2.9 Torr, at 25° C., about 0.6 Torr to about 1 Torr, at 25° C., less than, equal to, or greater than about 0.57 Torr, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, or about 2.9 Torr, at 25° C. Specific examples of suitable fragrance components include triplal, lemon oil, bergamot oil, pink pepper oil, ethyl acetate, benzaldehyde, Cis 3-hexenol, manzanate, eugenol, methyl anthranilate, benzyl acetate, dimethyl sulfide, ethyl butyrate, melonal, methyl pamplemousse, dihydromyrcenol, oxane, phenyl ethyl alcohol, methyl anthranilate, eugenol, galaxoildone, or a mixture thereof.

It has been surprisingly and unexpectedly found that including the carbon-emissions alcohol distillate in the composition has several benefits compared to a corresponding composition that includes conventionally produced ethanol. For example, as demonstrated herein in the Examples, including the carbon-emissions alcohol distillate can result in enhancing, improving or modifying an intensity, sensitivity or both of a perception of a fruity note, a citric note, a green note, a spicy note, or a mixture thereof in the disclosed composition. Additionally, it has been surprisingly and unexpectedly found that including the carbon-emissions alcohol distillate can reduce a pungency of ethanol perceived in a cosmetic, perfume, or both in an ethanol-based composition as compared to a corresponding cosmetic, perfume or both that includes conventionally prepared ethanol. Either of the aforementioned properties can be evaluated within 5 minutes of its production, withing 4 minutes of its production, within 3 minutes of its production, within 2 minutes of its production, or within 1 minute of its production.

In addition to the carbon-emissions alcohol distillate and fragrance component, the compositions described herein can include other ingredients such as colorants, UV-filters, antioxidants, thickeners, humectants, skin conditioners, emulsifiers, vitamins, chelatants, sensory enhancers, fragrance modulators and controlled-release ingredients.

### EXAMPLES

Various embodiments of the present disclosure can be better understood by reference to the following Examples which are offered by way of illustration. The present disclosure is not limited to the Examples given herein.

### Test Method 1: Sensory Testing-Triangle Test

At the testing facility, samples of the compositions and the controls are applied to glass slides (50 mm width) and placed on a hot plate at 32° C. to represent skin temperature during 2-3 minutes. It is important that glass slides of samples that are to be later compared are prepared at the same time.
Assessment of fragranced composition: Using a ‘Socorex’ regulated pipette, apply 20 μl of the fragranced composition carefully to glass slide allowing it to spread evenly.

Slides are presented randomized, coded with three digit numbers so that their identity is not known by the panelist. Three samples, two of which are the same, and one which is different are presented to the panelists in the same tray, and panelists are able to indicate which is the odd sample. Thirty-two panelists participated in experiment, which were screened for their sensory ability as a minimum requirement.

### Test Method 2: Shelf Life Evaluation and Ethanol Olfactive Evaluation

At least 3 expert evaluators were selected for these experiments. Panelists are asked to give a score on a scale of 1 to 5 for changes in the perceived fragrance profile change for the test compositions versus the controls according to the odor grading scale set out in Table 1 herein below. The median values are reported. Blotters are dipped in the samples and immediately after they are given to the evaluators.

Test samples for shelf life were evaluated at 8 weeks, 45° C. storage conditions, and 3 weeks 55° C. storage conditions.

TABLE 1
Character Difference Odour Grading Scale

| Score | Fragrance Profile change |
|---|---|
| 1 | Total difference in the fragrance profile from the control. |
| 2 | Large difference in fragrance profile from the control |
| 3 | Moderate fragrance profile but similar character to the |
| | control. Difference large enough for consumers to notice |
| | difference. |
| 4 | Slight fragrance profile change when compared directly |
| | with the control. |
| 5 | Fragrance profile is unchanged, i.e., no difference between |
| | the sample vs. the control. |

The controls for shelf-life testing were the same compositions kept at 4° C. to 6° C., thus considered unchanged. In the case of ethanol evaluation, both the control, which was the agricultural-based ethanol, and the test sample which was the carbon-based ethanol were kept at 4° C. before testing.

Furthermore, evaluator's comments were captured in order to distinguish if the carbon-emissions-based ethanol-containing product was considered better than the reference formulated with agricultural-crop-based ethanol.

### Test Method 3: Blooming Screening Evaluation

Five panelists who are all expert evaluators were selected for these experiments. Panelists are asked to perform a triangle test. Three samples, two of which were the controls, and one which are the formulated with carbon-based ethanol are presented to the panelists, and panelists are able to indicate which is the odd sample. They repeat this evaluation three times in order to ascertain the repeatability of their result. When at least 4 panelists were able to find out the odd sample repeatedly, it was considered easy to pick. When 3 panelists were able to pick difference, it was considered medium difference. Otherwise it was considered low difference. Blotters are dipped in the samples and immediately after they are given to the evaluators for testing.

### Test Method 4: Determining Vapor Pressure

In order to determine the vapor pressure for the fragrance materials, the software Advanced Chemistry Development (ACD/Labs) V11.02 (@ 1994-2013 ACD/Labs) was used. Perfume raw materials properties were predicted by using their structural formula. Vapor Pressure is expressed in 1 Torr, which is equal to 0.133 kilopascal (kPa).

### Example 1—Exemplary Product Compositions

Compositions 1-2 are examples of deodorants natural spray (“DNS”) and examples of after-shave lotions (“ASL”) respectively, and compositions 3-12 are examples of eau-de-toilette and eau-de-parfum (“EDT” and “EDP” respectively). According to the present invention, at least one ethanol and one fragrance composition need to be present in product. Other ingredients such as colorants, UV-filters, antioxidants, thickeners, humectants, skin conditioners, emulsifiers, vitamins, chelatants, sensory enhancers, fragrance modulators and controlled-release ingredients may be used in the formulation of these products.

TABLE 2
Formulated-fragrance compositions

| | | | Composition | Composition |
|---|---|---|---|---|
| | | | wt % | wt % |
| Composition | Type | Olfactive Character | Ethanol | Fragrance |
| 1 | DNS | Citrus/Musky | 70-80 | 1-5 |
| 2 | ASL | Aromatic/Citrus | 50-70 | 2-5 |
| 3 | EDT | Citrus/Musky | 70-80 | 5-15 |
| 4 | EDP | Floral/Citrus | 65-75 | 15-25 |
| 5 | EDT | Woody/Fruity | 70-80 | 5-15 |
| 6 | EDP | Floral/White Flower | 70-80 | 15-20 |
| 7 | EDP | Oriental/Fruity | 70-85 | 10-20 |
| 8 | EDP | Chypre/Floral | 75-80 | 15-25 |
| 9 | EDT | Fruity/Floral | 70-90 | 5-15 |
| 10 | EDP | Floral/Amber | 70-90 | 15-25 |
| 11 | EDP | Floral/Woody | 70-90 | 15-25 |
| 12 | EDP | Floral/Fruity | 70-90 | 15-25 |

The fragrance component includes at least one high volatile fragrance material. Compositions 13 to 31 were formulated in order to test the effect of alcohol in a simpler model. Perfume raw materials (“PRMs”) were introduced at a designated concentration in an 88.6:11.4 ethanol (96%): water solution.

TABLE 3
Perfume raw materials-compositions

| | | | | | Vapor |
|---|---|---|---|---|---|
| | | | | PRM | Pressure |
| | | Olfactive | | concentration | at 25° C. |
| Composition | PRM name | Character | Supplier | (wt %) | (mmHg) |
| 13 | Dimethyl Sulfide | Green | Sigma-Aldrich | 0.05-0.15 | 646.973 |
| 14 | Ethyl Acetate | Fruit | Essencia | 0.05-0.15 | 111.716 |
| 15 | Ethyl Butyrate | Fruit | Symrise | 0.01-0.04 | 13.941 |
| 16 | Manzanate | Fruit | Givaudan | 0.02-0.07% | 2.906 |
| 17 | Pink Pepper Oil | Spicy | Firmenich | 0.10-0.30 | 1.856^{a} |
| 18 | Lemon Oil Verdelli Italy | Citrus | Simone Gatto | 0.50-0.90 | 1.541^{b} |
| 19 | Bergamot Oil Italy | Citrus | Simone Gatto | 0.50-1.5 | 1.541^{b} |
| 20 | Hexyl Acetate | Fruit | Symrise | 0.20-0.40 | 1.391 |
| 21 | Hexenol cis-3 | Green | Ventos | 0.02-0.06 | 1.039 |
| 22 | Benzaldehyde | Fruit | Symrise | 0.01-0.05 | 0.974 |
| 23 | Melonal | Fruit | Symrise | 0.02-0.08 | 0.622 |
| 24 | Triplal | Green | IFF | 0.03-0.07 | 0.578 |
| 25 | Methyl Pamplemousse | Citrus | Essencia | 0.3-0.7 | 0.214 |
| 26 | DyhydroMyrcenol | Citrus | Essencia | 0.3-0.7 | 0.166 |
| 27 | Benzyl Acetate | Floral | Essencia | 0.3-0.7 | 0.164 |
| 28 | Oxane | Fruit | Firmenich | 0.02-0.07 | 0.123 |
| 29 | Phenyl Ethyl Alcohol | Floral | Firmenich | 0.5 to 1.5 | 0.0741 |
| 30 | Methyl Anthranilate | Fruity | Essencia | 0.03-0.07 | 0.0158 |
| 31 | Eugenol | Spicy | Essencia | 0.05-0.15 | 0.0104 |

^{a}This is a multicomponent ingredient where alpha-phellandrene is the main ingredient
^{b}This is a multicomponent ingredient where Limonene is the main ingredient

### Example 2—Shelf-Life Evaluation and Comparison

Fragrance Compositions disclosed in Tables 2 and 3 were tested in accordance with the protocol described in the Test Method Section 2 and a panel of at least 3 expert evaluators assess the perceived fragrance profile at initial time 0. Panelists are asked to score the compositions for the fidelity of the fragrance profile on a scale of 1 to 5, wherein 5 represents a no fragrance character change is detected and 1 represents a complete fragrance character change detected versus the sample using the same alcohol kept at 4° C. The median of the results of the panelists is used and discussed below. Furthermore, panelists were asked to comment whether the fragrance compositions formulated with the carbon-based ethanol were better, the same or worse perceived than the reference made with agricultural-based ethanol.

Table 4 shows the effect on shelf life of the carbon-emissions-based ethanol-containing products versus the reference products containing agricultural-based ethanol. Most compositions degrade in a similar matter when comparing against a control at 4° C. However, out of 12 formulations containing carbon-emissions-based ethanol, 7 were perceived as better and 5 were perceived as not changing compared to the reference product. Analyzing the oxidative markers, in the case of composition 1, 5, 6, 9, 11, and 12, the levels of Diethylacetal Acetaldehye (“DEAA”) were reduced in ranges from 42-75% for those formulas containing carbon-emissions-based ethanol. Additionally, the level of BHT, a well-known antioxidant added in fragrances, remained 32% and 24% higher in composition 5 and 6 respectively, indicating less oxidation. DEAA is a product generated by reactions in ethanol which brings a moldy, grappa note—considered unpleasant. In the case of composition 6, even though expert panelists considered it comparable olfactively, the slightly yellower aspect in the sample formulated with agricultural ethanol and higher oxidative markers after 3 weeks and 8 weeks (data not shown), indicated more oxidation. Similar results were found for composition 9 where samples formulated with agricultural ethanol were slightly more pink, and more oxidized after 3 weeks and 8 weeks. Other oxidation markers like Galaxidone, and Linalool Oxide, present at higher quantities in the perfumes formulated with agricultural ethanol, are product of oxidation.

TABLE 4
Evaluation of olfactive character over
shelf life and oxidative markers

| | | Expert | | |
|---|---|---|---|---|
| | Shelf Life | Evaluation | | |
| | Evaluation | Carbon- | Evaluator | Oxidative |
| | Agricultural | Emission | comment | markers |
| Compo- | Ethanol - | Ethanol - | vs. ref - | difference - |
| sition | 8 w 45° C. | 8 w 45° C. | 8 w 45° C. | 3 w 55° C. |
| 1 | 3 | 3 | + | DEAA −75% |
| 2 | 4 | 4 | + | |
| 3 | 3 | 3 | + | |
| 4 | 3 | 3 | 0 | |
| 5 | 3 | 3 | + | DEAA −73% |
| | (borderline) | | | Linalool |
| | | | | Oxide −33% |
| | | | | BHT +32% |
| 6 | 3 | 3 | 0 | DEAA −43% |
| | | | | BHT +24% |
| 7 | 3 | 3 | 0 | |
| 8 | 4 | 4 | 0 | |
| 9 | 4 | 4 | 0 | DEAA −48% |
| 10 | 3 | 3 | + | |
| 11 | 3 | 3 | + | DEAA −62% |
| | | | | Galaxolidone −37% |
| 12 | 3 | 3 | + | DEAA −42% |

### Example 3—Bloom Character Change in Formulations and PRMs, and Consumer Triangle Test

Table 5 below shows the impact of the different ethanol's on blooming olfactive character for a variety of fully formulated perfumes (compositions 1-12) and for PRM's (composition 13-31). Unsurprisingly, the differences due to the ethanol type are more easily perceived in full formulations than individual raw materials due to the potential multiple possible interactions with the ethanol. 60% of formulations were considered easy-to-pick versus 20% in the case of PRM's. A similar trend was observed when performing triangle tests with consumers, where 2 out of the 3 formulations were perceived significantly different (p<0.05) whereas only 1 out of 3 of the PRM's were perceived significantly different (p<0.10). The later result is more likely due to the time of testing, as the easy-to-pick PRM's were very volatile and at lower concentrations than the lemon oil, and thus could have made the comparison difficult for consumers. As well the full formulations had higher content of perfume raw materials and solvents alike, probably affecting the volatility of ethanol as well-which meant that at 3 minutes, the impact of ethanol was easier perceived in the full formulations.

In general, it can be seen that perfumes and PRM's containing the carbon-based ethanol are more likely to appear fresher, greener, more citrus, more fruity, and less floral. This is non-excluding, as other experiments have remarked enhanced characters like patchouli and cedarwood (data not shown). PRM's with a vapor pressure in a range of 0.57 Torr and 2.9 Torr, and more particularly from 0.6 and 1 Torr, have higher tendency to show differences based on ethanol type, with the exception of Hexyl Acetate. Vapor pressures above and below those values seem less likely to be affected by ethanol type.

TABLE 5
Evaluation of fragrance bloom by experts and consumer's triangle test

| | Blooming Test - | | Triangle Test - |
|---|---|---|---|
| | Expert Evaluator - | | Consumers - |
| Composition | t = 0 min | Olfactive description - Experts | p-value |
| 1 | Easy to pick | More anis star, more brighter, more | 0.144 |
| | | transparent, more clean | |
| 2 | Easy to pick | More citrus, fresh aromatic | 0.038 |
| 3 | Easy to pick | More citrus, green, lime, less floral, strong | — |
| | | distortion of the note | |
| 4 | Medium | Greener, less floral sweet | — |
| 5 | Easy to pick | Less leathery, more apple, more vanilla | — |
| 6 | Easy to pick | Greener, hexanol, more jasmine, indol, | 0.038 |
| | | less tuberose | |
| 7 | Medium | More mouldy, patchouli on this first | — |
| | | feeling, more pineapple juicy after | |
| 8 | Medium | More plum, more fruity | — |
| 9 | Medium | Greener, less fruity, cis-3 -hexenol | — |
| 13 | Low | — | — |
| 14 | Medium | More fruity, less grappa | — |
| 15 | Low | — | — |
| 16 | Medium | Greener, less strawberry, | — |
| 17 | Medium | More long-lasting, more caryophyllene | — |
| 18 | Medium | More juicy, greener, long-lasting | 0.1 |
| 19 | Medium | More lemon, more powerful, fresh | — |
| 20 | Low | — | — |
| 21 | Easy to pick | More fruity, apple, long-lasting, pure | 0.821 |
| 22 | Easy to pick | Different on cherry side, long-lasting | — |
| 23 | Easy to pick | More fruity, melon, more mineral, aldehydic | 0.821 |
| 24 | Medium | Less chemical, more hexenol, fresh cut grass | — |
| 25 | Low | — | — |
| 26 | Low | — | — |
| 27 | Medium | More rounded, more fruity, more banana, less | — |
| | | phenolic, more floral and soft, less rubbery, | |
| | | less solvent. | |
| 28 | Low | — | — |
| 29 | Low | — | — |
| 30 | Medium | Less floral, very phenolic | — |
| 31 | Easy to pick | More clove, more natural, round, less leathery, | — |
| | | more powerful, more peppery, less diffusing, | |

### Example 4—Ethanol Olfactive Description

The carbon-based ethanol was compared against two different agricultural-based ethanol on blotter. Results can be seen below on Table 6, showing that overall, both types of ethanol are comparable but have slight notes differences. All comments from expert evaluators seem to indicate that the carbon-based ethanol is perceived more neutral. As well, the comments of less dusty, less pungent and less “alcohol” are in line with previously described benefits in literature.

TABLE 6
Olfactive evaluation comparison between agricultural-based ethanol
(reference) and carbon-captured-based ethanol on blotters

| | | Carbon-captured-based |
|---|---|---|
| Evaluation | Scores | ethanol comments vs. Ref |
| Undenatured Carbon- | 4 - 4 - 3 - Median: 4 | Less fusing, less dusty, |
| captured Ethanol | | more volatile, Carbon- |
| | | based ethanol better, |
| | | flatter, comparable |
| Denatured Carbon- | 4 - 4 - 3 - Median: 4 | Less smelly, less alcohol, |
| Captured Ethanol | | less dusty, less pungent, |
| | | less lift, powdery, |
| | | slightly less earthy |

### Exemplary Embodiments

The following exemplary embodiments are provided, the numbering of which is not to be construed as designating levels of importance:

Aspect 1 provides a composition comprising:
a carbon-emissions-derived ethanol distillate; and
a fragrance component,
wherein the carbon-emissions-derived ethanol distillate is a distillate comprising ethyl alcohol and the composition is formulated as a cosmetic or a perfume.

Aspect 2 provides the composition of Aspect 1, wherein the carbon-emissions-derived ethanol distillate further comprises volatile and non-volatile by-products.

Aspect 3 provides the composition of any one of Aspects 1 or 2, wherein the composition comprises from about 50% to about 95% of the carbon-emissions alcohol distillate.

Aspect 4 provides the composition of any one of Aspects 1-3, wherein the composition comprises from about 70% to about 95% of the carbon-emissions alcohol distillate

Aspect 5 provides the composition of any one of Aspects 1˜4 wherein said carbon-emissions ethanol distillate is a fermentation product.

Aspect 6 provides the composition of any one of Aspects 1-5 wherein the carbon-emissions comprise CO, CO₂, H₂, or a mixture thereof.

Aspect 7 provides the composition of any one of Aspects 1-6, wherein the composition comprises about 0.1 to about 30% of the fragrance component.

Aspect 8 provides the composition of any one of Aspects 1-7, wherein the composition comprises about 5 to about 25% of the fragrance component.

Aspect 9 provides the composition of any one of Aspects 1-8, wherein the fragrance component comprises a material having an olfactive character classified as a green note, a fruity note, a spicy note, a citrus note, a floral note, or a combination thereof.

Aspect 10 provides the composition of any one of Aspects 1-9, wherein the fragrance component comprises at least one volatile fragrance material having a vapor pressure in a range of from about 0.57 Torr to about 2.9 Torr, at 25° C.

Aspect 11 provides the composition of Aspect 10, wherein the fragrance component comprises at least one volatile fragrance material having a vapor pressure in the range of from about 0.6 Torr to about 1 Torr, at 25° C.

Aspect 12 provides the composition of any one of Aspects 1-11, wherein the fragrance component is selected from the group comprising triplal, lemon oil, bergamot oil, pink pepper oil, ethyl acetate, benzaldehyde, Cis 3-hexenol, manzanate, eugenol, methyl anthranilate, benzyl acetate, dimethyl sulfide, ethyl butyrate, melonal, methyl pamplemousse, dihydromyrcenol, oxane, phenyl ethyl alcohol, methyl anthranilate, eugenol, galaxoilde, linalool or a mixture thereof.

Aspect 13 provides the composition of any one of Aspects 1-12, wherein said composition is formulated as a perfume in a form selected from the group comprising an extract, eau de toilette, a creamy perfume, a bath preparation, an environmental fragrance, or a mixture thereof.

Aspect 14 provides the composition of Aspect 13, wherein the perfume is in a liquid form, a gelled form, a viscous form, solid form, or a mixture thereof.

Aspect 15 provides a method of making the composition of any one of Aspects 1-14, the method comprising:
fermenting a carbon emission to produce a carbon-emissions-derived ethanol composition;
distilling the carbon-emission-derived ethanol composition to produce a carbon-emissions-derived ethanol distillate; and
mixing the carbon-emissions-derived ethanol distillate with the fragrance component.

Aspect 16 provides the method of Aspect 15, wherein the carbon emission is fermented using an acetogenic bacteria.

Aspect 17 provides a method for enhancing, improving or modifying an intensity, sensitivity or both of a perception of a fruity note, a citric note, a green note, a spicy note, or a mixture thereof of the fragrance component, comprising preparing the composition of any one of Aspects 1-16.

Aspect 18 provides the method of Aspect 17, wherein the intensity, sensitivity, or both of the perception of the fruity note, the citric note, the green note, the spicy note, or the mixture thereof of the fragrance component is enhanced, improved, or modified compared to a corresponding fragrance composition differing in that the ethyl alcohol in the corresponding fragrance composition is not a carbon-emissions-derived ethanol distillate.

Aspect 19 provides a method for reducing a pungency of ethanol perceived in a cosmetic, perfume, or both in an ethanol-based composition, comprising preparing the composition of any one of Aspects 1-18.

Aspect 20 provides the method of Aspect 19, wherein the pungency of ethanol perceived in a cosmetic, perfume, or both in the ethanol-based composition, is reduced compared to a corresponding fragrance composition differing in that the ethyl alcohol in the corresponding fragrance composition is not a carbon-emissions-derived ethanol distillate.

Aspect 21 provides the method of any one of Aspects 19 or 20, wherein the cosmetic or perfume is evaluated before 5 minutes.

Aspect 22 provides the method of any one of Aspects 19-21, wherein the cosmetic or perfume is evaluated before 2 minutes.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A composition comprising:
a carbon-emissions-derived ethanol distillate; and
a fragrance component selected from the group comprising triplal, lemon oil, bergamot oil, pink pepper oil, ethyl acetate, benzaldehyde, Cis 3-hexenol, manzanate, eugenol, methyl anthranilate, benzyl acetate, dimethyl sulfide, ethyl butyrate, melonal, methyl pamplemousse, dihydromyrcenol, oxane, phenyl ethyl alcohol, methyl anthranilate, eugenol, galaxoilde, linalool or a mixture thereof,
wherein the carbon-emissions-derived ethanol distillate is a sufficiently distilled distillate comprising ethyl alcohol and the composition is formulated as a cosmetic or a perfume; and
the composition exhibits greater stability and has a less perceivable ethanol odor than a corresponding composition differing only in that the corresponding composition comprises agriculturally derived ethanol, a fruit fermented ethanol, or both
wherein said carbon-emissions ethanol distillate is a fermentation product.

25. The composition of claim 24, wherein the carbon-emissions-derived ethanol distillate further comprises volatile and non-volatile by-products.

26. The composition of claim 24, wherein the composition comprises from about 50% to about 95% of the carbon-emissions alcohol distillate.

27. The composition of claim 24, wherein the composition comprises from about 70% to about 95% of the carbon-emissions alcohol distillate

28. The composition of claim 24, wherein said carbon-emissions ethanol distillate is a fermentation product.

29. The composition of claim 24, wherein the carbon-emissions comprise CO, CO2, H2, or a mixture thereof.

30. The composition of claim 24, wherein the composition comprises about 0.1 to about 30% of the fragrance component.

31. The composition of claim 24, wherein the composition comprises about 5 to about 25% of the fragrance component.

32. The composition of claim 24, wherein the fragrance component comprises a material having an olfactive character classified as a green note, a fruity note, a spicy note, a citrus note, a floral note, or a combination thereof.

33. The composition of claim 24, wherein the fragrance component comprises at least one volatile fragrance material having a vapor pressure in a range of from about 0.57 Torr to about 2.9 Torr, at 25° C.

34. The composition of claim 33, wherein the fragrance component comprises at least one volatile fragrance material having a vapor pressure in the range of from about 0.6 Torr to about 1 Torr, at 25° C.

35. The composition of claim 24, wherein the composition is free of a vinic alcohol.

36. The composition of claim 24, wherein said composition is formulated as a perfume in a form selected from the group comprising an extract, eau de toilette, a creamy perfume, a bath preparation, an environmental fragrance, or a mixture thereof.

37. The composition of claim 36, wherein the perfume is in a liquid form, a gelled form, a viscous form, solid form, or a mixture thereof.

38. The composition of claim 24, wherein the perception of an oxidative marker is diminished relative to that of a corresponding composition including an agriculturally derived ethanol.

39. The composition of claim 38, wherein the oxidative marker comprises diethyl acetal cetaldehye, butylated hydroxytoluene, galaxoilde, linalool or a mixture thereof,

40. A method of making the composition of claim 24, the method comprising:
fermenting a carbon emission to produce a carbon-emissions-derived ethanol composition;
distilling the carbon-emission-derived ethanol composition to produce a carbon-emissions-derived ethanol distillate; and
mixing the carbon-emissions-derived ethanol distillate with the fragrance component.

41. The method of claim 40, wherein the carbon emission is fermented using an acetogenic bacteria.

42. A method for enhancing, improving or modifying an intensity, sensitivity or both of a perception of a fruity note, a citric note, a green note, a spicy note, or a mixture thereof of the fragrance component, comprising preparing the composition of claim 24.

43. The method of claim 42, wherein the intensity, sensitivity, or both of the perception of the fruity note, the citric note, the green note, the spicy note, or the mixture thereof of the fragrance component is enhanced, improved, or modified compared to a corresponding fragrance composition differing in that the ethyl alcohol in the corresponding fragrance composition is not a carbon-emissions-derived ethanol distillate.
